(19) United States
(12) Patent Application Publication
(10) Pub. No.: US 2025/0152395 A1
(43) Pub. Date: May 15, 2025

(54) WRIST AND ELBOW STABILIZING REHABILITATION ARM BRACE

(71) Applicant: Jacob T Moylan, Shawnee, KS (US)

(72) Inventor: Jacob T Moylan, Shawnee, KS (US)

(21) Appl. No.: 18/943,397

(22) Filed: Nov. 11, 2024

Related U.S. Application Data
(60) Provisional application No. 63/548,097, filed on Nov. 10, 2023.

(51) Int. Cl. A61F 5/01

(52) U.S. Cl. CPC A61F 5/013; A61F 2005/0158; A61F 2005/0167

(57) ABSTRACT

A stabilizing rehabilitation arm brace includes a proximal anchor assembly, a distal anchor assembly and an extension bar connecting same. A method of stabilizing and rehabilitating a patient's arm includes the steps of placing a brace on the upper arm and wrist for restricting movement and enabling recovery.

### CROSS-REFERENCE TO RELATED APPLICATION

This application claims priority in U.S. Provisional Patent Application Nos. 63/548,097, filed on Nov. 10, 2023, which is incorporated herein by reference.

### BACKGROUND OF THE INVENTION

### 1. Field of the Invention

The present invention relates generally to an arm brace, and more specifically to a wrist and elbow stabilizing rehabilitation arm brace.

### 2. Description of the Related Art

Treatment protocols for muscular-skeletal injuries and other conditions commonly involve restricting normal movements. For example, fractures and sprains are normally isolated and the adjacent limbs are typically restrained in casts and slings. With the affected joints immobilized, normal healing can proceed and patient discomfort is minimized. Conversely, reinjuring an existing condition before complete healing is achieved can delay progress and compromise patient recovery.

A wide variety of braces have been introduced and commercialized for specific conditions. For example, wrists and elbows are susceptible to fractures and sprains from physical activity, including falls and impact forces. Such injuries are particularly challenging because active patients constantly utilize their shoulders, arms and hands for a wide range of tasks. Simple tasks can produce significant pain and reinjury if appropriate precautions are not taken. Rigid casts and splints, however, can be overly restrictive and compromise activity and quality of life. Moreover, immobility can lead to muscle atrophy, further delaying rehabilitation and full-functional restoration.

Patients in physically demanding occupations are particularly impacted adversely by such functionally compromising conditions. Manual workers, athletes, first responders, healthcare workers and others may be unable to fully perform their respective tasks while recovering. Mobility and recreational pursuits are also restricted and impaired.

Heretofore there has not been available a system or method for a wrist and elbow stabilizing rehabilitation arm brace with the advantages and features of the present invention.

### BRIEF SUMMARY OF THE INVENTION

The present invention generally provides a rehabilitation arm brace that restrains the wrist from rotating and prevents the elbow from bending. This allows the user to protect his or her injured arm during rehabilitation while enabling some useful function of the hand and arm. The rehabilitation brace is adjustable for accommodating individuals of different sizes and adjusting restrictive movements. Attachment and removal can be quickly and efficiently accomplished using common connecting devices where appropriate. Moreover, the restraining brace components that engage patient are configured for optimal patient comfort and convenience.

### BRIEF DESCRIPTION OF THE DRAWINGS

The drawings constitute a part of this specification and include exemplary embodiments of the present invention illustrating various objects and features thereof.

FIG. 1 is a side view of a wrist and elbow stabilizing rehabilitation arm brace embodying an aspect of the present invention, shown on the right arm of an individual.

FIG. 2 is a front view of the brace on the individual.

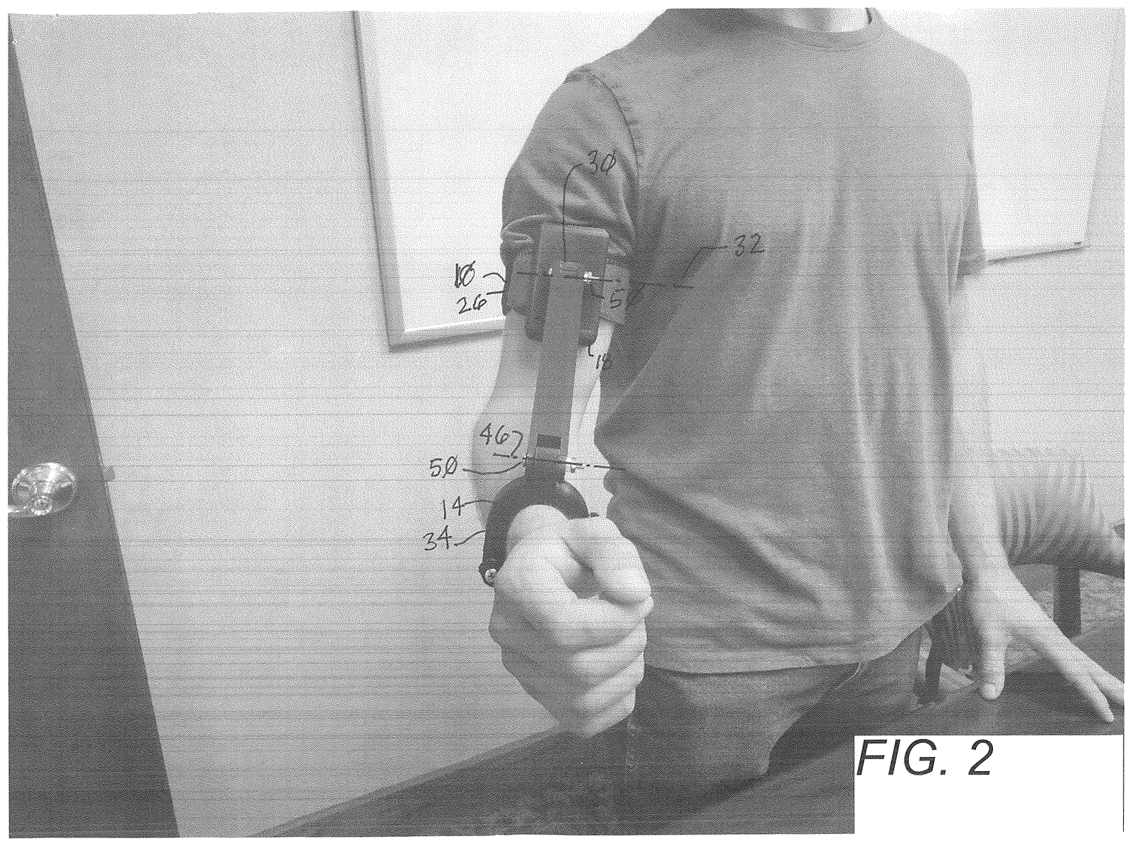

FIG. 3 is a front view of the brace with the braced arm extended.

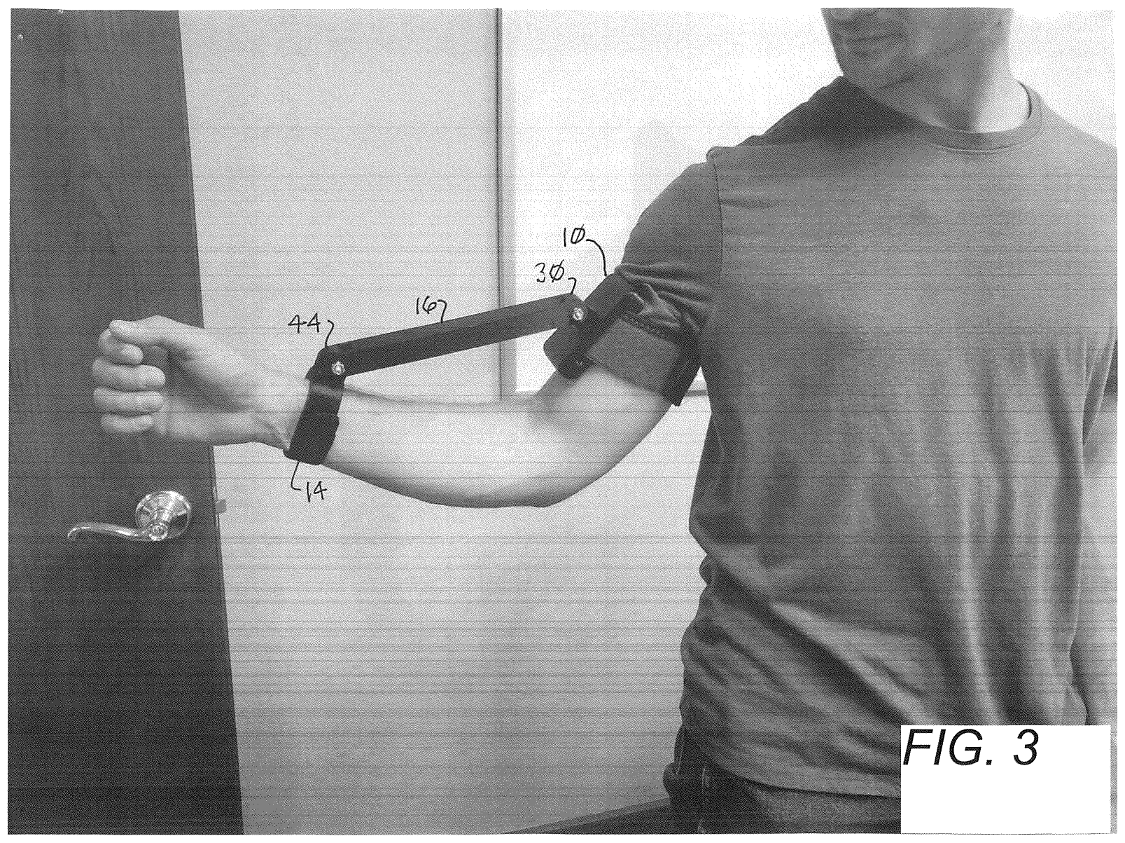

FIG. 4 is a perspective view of the race with a wrist restraint in an open position.

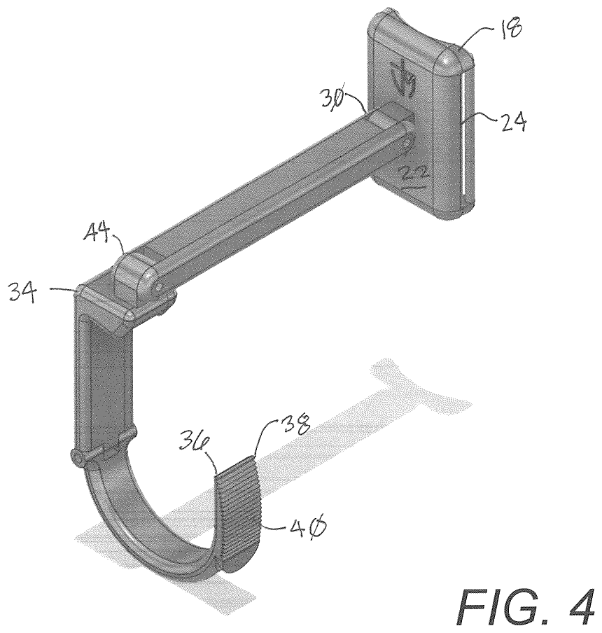

FIG. 5 is a perspective view of an underside of the brace. An optional extension arm outer sleeve is shown in dashed lines and telescopically receives an extension arm inner tube for adjusting the extension arm length.

FIG. 6 is an exploded, perspective view showing the brace components disassembled.

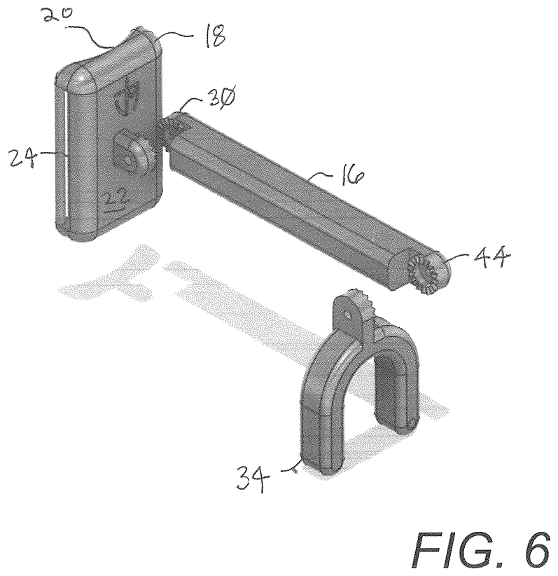

FIG. 7 is a perspective view of a bicep pad of the brace.

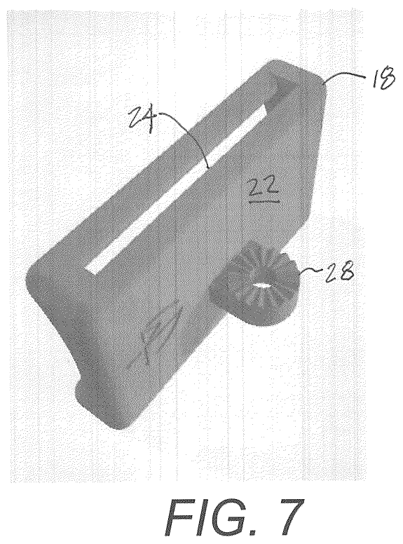

FIG. 8 is another perspective view of the bicep pad.

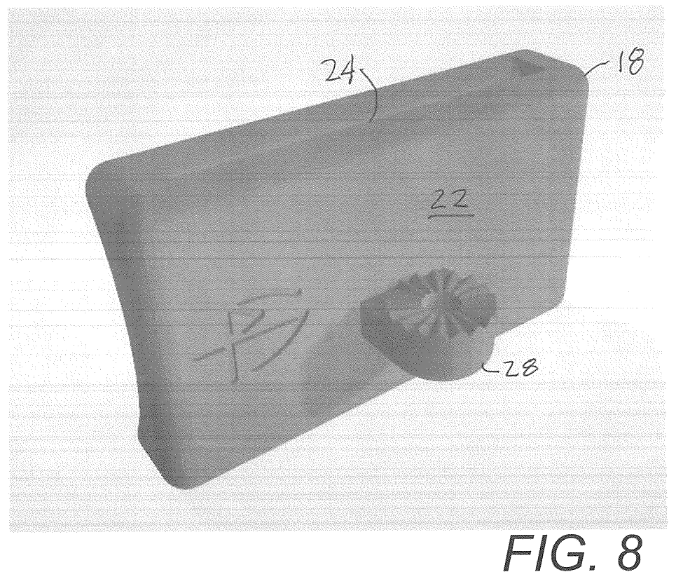

FIG. 9 is a perspective view of an extension bar of the brace.

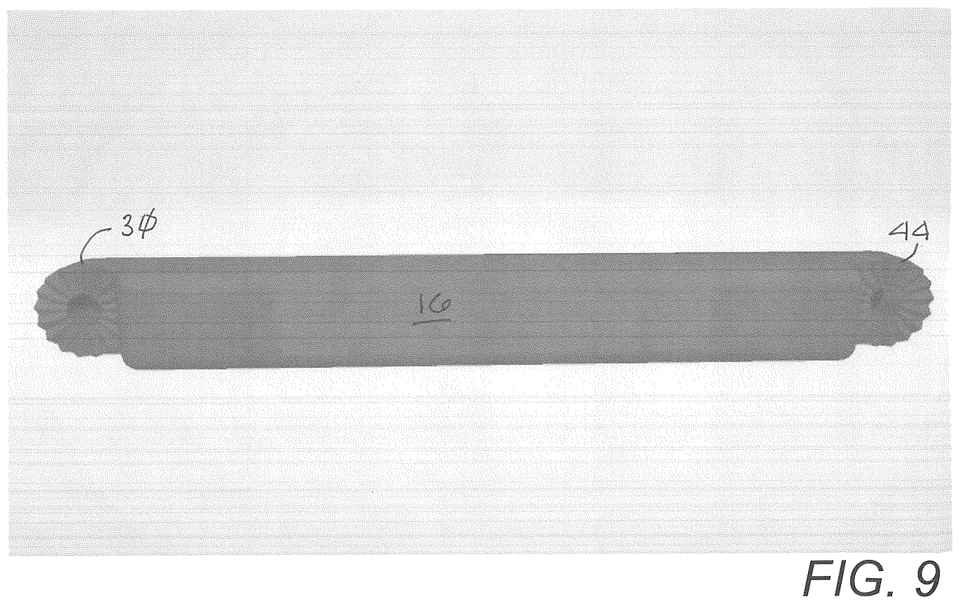

FIG. 10 is a perspective view of a wrist restraint of the brace.

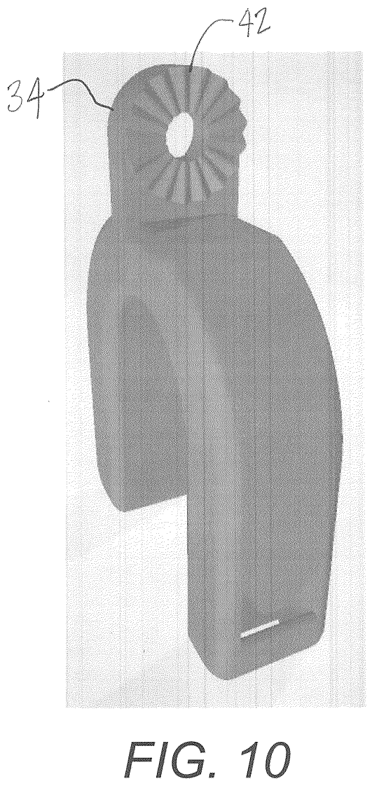

FIG. 11 is a perspective view of a toggle clamp configured for releasably connecting ends of the extension bar to the bicep pad and the wrist restraint.

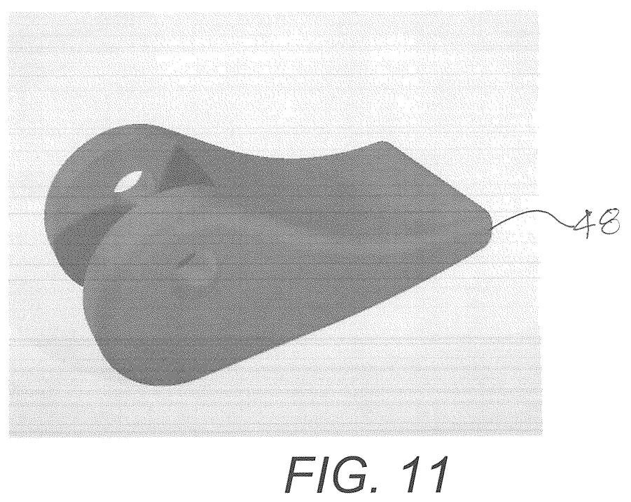

FIG. 12 is a perspective view of the wrist restraint in a closed position.

FIG. 13 is a perspective view of the wrist restraint in an open position.

### DETAILED DESCRIPTION OF THE PREFERRED EMBODIMENTS

### I. Introduction and Environment

As required, detailed aspects of the present invention are disclosed herein, however, it is to be understood that the disclosed aspects are merely exemplary of the invention, which may be embodied in various forms. Therefore, specific structural and functional details disclosed herein are not to be interpreted as limiting, but merely as a basis for the claims and as a representative basis for teaching one skilled in the art how to variously employ the present invention in virtually any appropriately detailed structure.

Certain terminology will be used in the following description for convenience in reference only and will not be limiting. For example, up, down, front, back, right and left refer to the invention as orientated in the view being referred to. The words, “inwardly” and “outwardly” refer to directions toward and away from, respectively, the geometric center of the aspect being described and designated parts thereof. Said terminology will include the words specifically mentioned, derivatives thereof and words of similar meaning.

### II. Preferred Embodiment Wrist and Elbow Stabilizing Rehabilitation Arm Brace

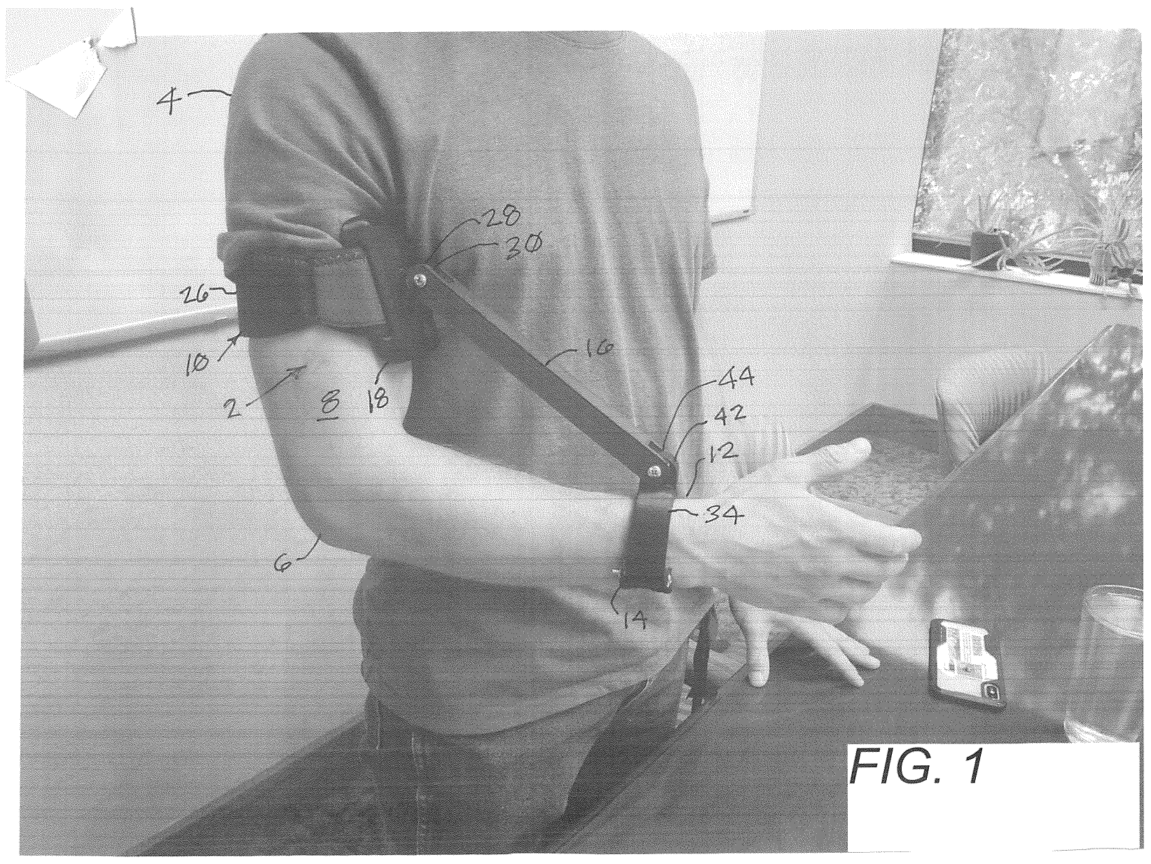

Referring to the drawings in more detail, FIG. 1 shows a stabilizing rehabilitation arm brace 2 embodying an aspect of the present invention and placed on a patient 4 for stabilizing and restraining the right arm 6. More specifically, the brace 2 is anchored on the upper arm 8 generally over the bicep by a proximal anchor assembly 10 and captures the wrist 12 with a distal anchor assembly 14. An extension bar 16 is connected to and extends between the anchor assemblies 10, 14.

The proximal anchor assembly 10 includes a bicep pad 18 with a concave inner face 20 and an outer face 22. A pair of slots 24 are provided on opposite sides of the bicep pad 18 and slidably receive a proximal anchor strap 26, which can be provided with hook-and-loop fasteners (e.g., Velcro® fasteners). Alternatively, the anchor strap 26 can utilize buckles, snaps, laces, or any other suitable fastening mechanism.

A proximal anchor tab 28 extends forwardly from the bicep pad outer face 22 and is pivotally mounted on an extension bar proximal end 30 for accommodating pivotal movement relative to a transverse, pivotal axis 32, i.e. generally parallel to the elbow pivotal axis.

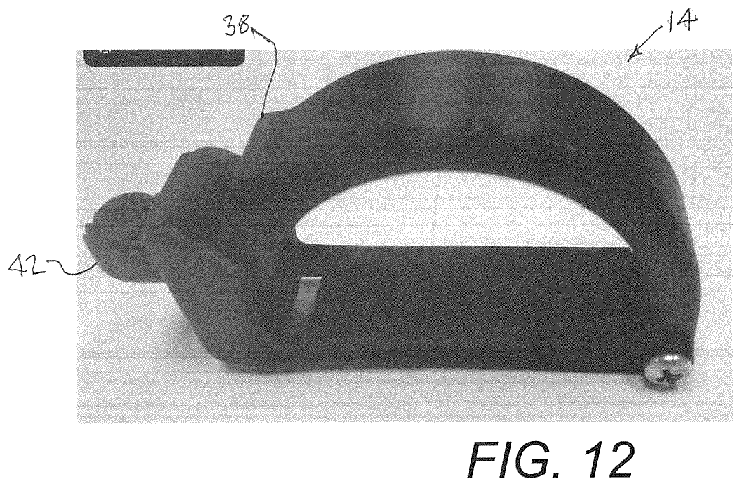

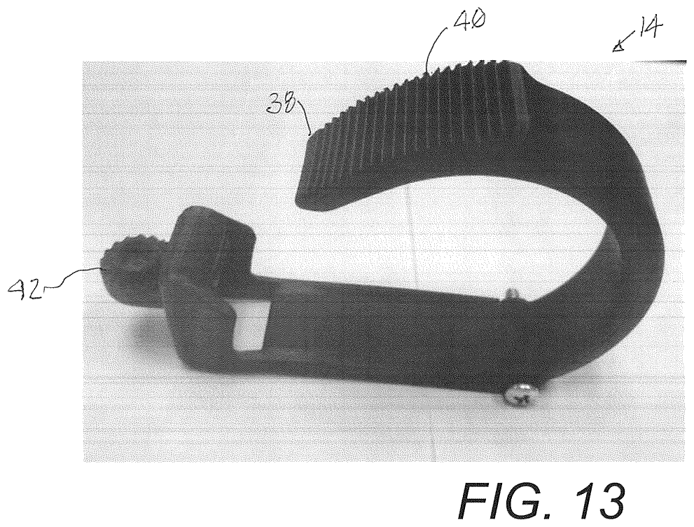

The distal anchor assembly 14 generally includes a cuff 34 configured for receiving and restraining the wrist 12 with a cuff restraint, 36, having open and closed configurations as shown in FIGS. 12 and 13 respectively. Without limitation on the generality of useful fasteners, a ratcheting fastener 38 is shown with teeth 40 whereby the cuff opening can be adjusted to securely restrain the wrist 12 and restrict rotation.

A distal anchor tab 42 extends generally upwardly from the cuff 34 and pivotally mounts an extension bar distal end 44 for limited rotational adjustment relative to a transverse axis 46, which is generally parallel to the proximate anchor pivotal axis 32. The extension bar 16 connections to the proximal and distal anchor tabs 28, 42 can be constructed with Hirth-type fasteners as shown, which include interlocking teeth for restricting pivotal movement when the components are bolted together. Quick-release lock 48 can be placed on connecting bolts 50 for expeditiously opening the connections to accommodate adjusting the relative positions and orientations of the bicep pad 18, the cuff 34 and the extension bar 16.

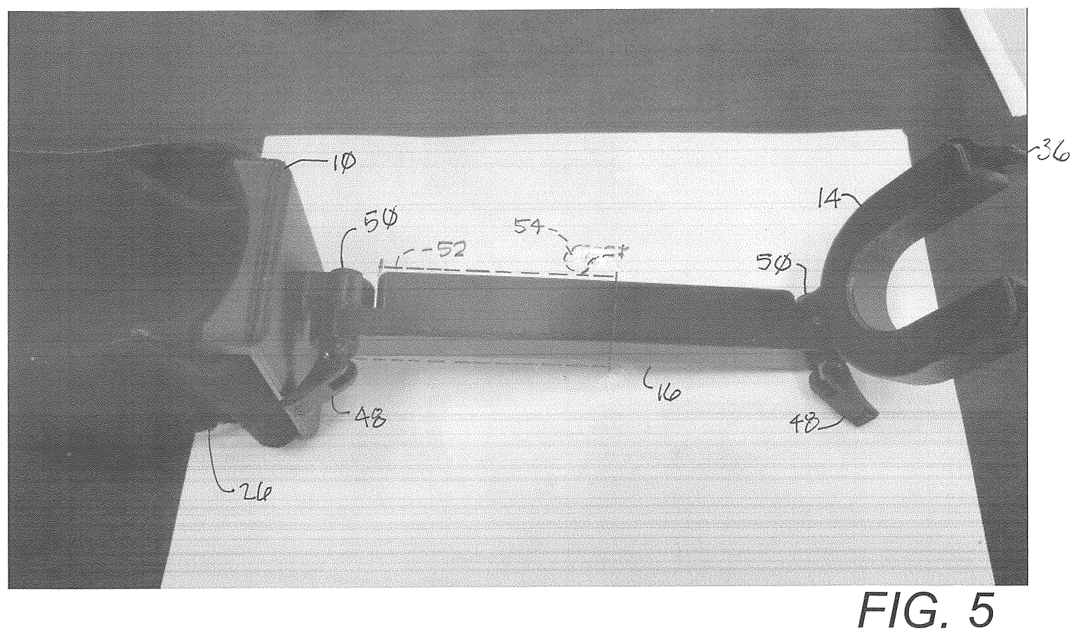

As shown in FIG. 5, the extension bar 16 can comprise inner and outer telescoping sections to accommodate length adjustment between the proximal anchor assembly 10 and the distal anchor assembly 14. An outer tubular sleeve 52 is shown in dashed lines. An extension bar locking mechanism 54 can be provided for locking the extension bar 12 at desired lengths.

It should further be noted that the invention may be made of plastic, silicone, metal, wood, ceramic, or other material. It may be made via 3D printing, milling, sintering, stamping, bending, casting, forging, injection molding, or other means.

It is to be understood that while certain embodiments and/or aspects of the invention have been shown and described, the invention is not limited thereto and encompasses various other embodiments and aspects.

Having thus described the invention, what is claimed as new and desired to be secured by Letters Patent is:

1. A stabilizing rehabilitation arm brace system comprising:
a proximal anchor assembly configured for placement on a patient's upper arm generally over the bicep;
a distal anchor assembly configured for placement on the patient's wrist; and
an extension arm pivotally connected to said proximal and distal anchor assemblies and configured for maintaining a predetermined distance therebetween.

2. The stabilizing brace according to claim 1 wherein said proximal anchor, said distal anchor and said extension arm are configured for restricting elbow and wrist movement.

3. The stabilizing brace according to claim 1, which includes:
said proximal anchor assembly including a bicep pad with an inner face engaging the upper arm over the bicep, an outer face and a proximal anchor tab extending from said bicep pad outer face; and
said proximal anchor assembly including a cuff configured for placement over the wrist, a cuff restraint with an open position for placing the cuff over the wrist and a closed position restraining said wrist in the cuff and a distal anchor tab extending from the cuff.

4. The stabilizing brace according to claim 1, which includes:
a proximate connecting bolt pivotally connecting said proximate anchor tab and said extension bar proximate end; and
a distal anchor bolt pivotally connecting said distal anchor tab and said extension bar, distal end.

5. The stabilizing brace according to claim 4, which includes:
proximate and distal quick-release tabs mounted on said proximate and distal connecting bolts respectively; and
each said quick-release tab configured for clamping a respective proximate and distal tab to a respective proximate and distal extension bar end.

6. The stabilizing brace according to claim 1, which includes:
said proximal anchor assembly bicep pad, including a pair of slots extending through opposite sides of said bicep pad; and
said proximal anchor assembly including a proximal anchor strap extending through said slots and configured for encircling the upper arm.

7. The stabilizing brace according to claim 6, wherein said proximal anchor strap includes a fastener for fastening on itself utilizing one of: hook-and-loop fasteners; snap fasteners; and buckle fasteners.

8. The stabilizing brace according to claim 1, which includes:
said extension bar comprising inner and outer telescoping tubular members;
said outer telescoping tubular member comprising an outer arm sleeve; and
an extension bar locking mechanism comprising a quick-release lock mounted on said outer telescoping member and having an open position enabling telescopic movement of said inner and outer members for adjusting a length of said extension bar and a closed position retaining said extension bar in a predetermined length.

9. The stabilizing brace according to claim 1 wherein said distal anchor assembly cuff restraint includes a ratcheting fastener with multiple teeth for adjusting a distal anchor assembly cuff restraint opening size and retaining said cuff on the wrist.

10. A stabilizing rehabilitation arm brace, which includes:
a proximal anchor assembly configured for placement on a patient's upper arm generally over the bicep;
a distal anchor assembly configured for placement on the patient's wrist;
an extension arm pivotally connected to said proximal and distal anchor assemblies and configured for maintaining a predetermined distance therebetween
said distal anchor and said extension arm are configured for restricting elbow and wrist movement;
said proximal anchor assembly including a bicep pad with an inner face engaging the upper arm over the bicep, an outer face and a proximal anchor tab extending from said bicep pad outer face;
said proximal anchor assembly including a cuff configured for placement over the wrist, a cuff restraint with an open position for placing the cuff over the wrist and a closed position restraining said wrist in the cuff and a distal anchor tab extending from the cuff;
a proximate connecting bolt pivotally connecting said proximate anchor tab and said extension bar proximate end;
a distal anchor bolt pivotally connecting said distal anchor tab and said extension bar, distal end;
proximate and distal quick-release tabs mounted on said proximate and distal connecting bolts respectively;
each said quick-release tab configured for clamping a respective proximate and distal tab to a respective proximate and distal extension bar end;
said proximal anchor assembly bicep pad, including a pair of slots extending through opposite sides of said bicep pad;
said proximal anchor assembly including a proximal anchor strap extending through said slots and configured for encircling the upper arm;
said proximal anchor strap including a fastener for fastening on itself utilizing one of: hook-and-loop fasteners; snap fasteners; and buckle fasteners;
said extension bar comprising inner and outer telescoping tubular members;
said outer telescoping tubular member comprising an outer arm sleeve;
an extension bar locking mechanism comprising a quick-release lock mounted on said outer telescoping member and having an open position enabling telescopic movement of said inner and outer members for adjusting a length of said extension bar and a closed position retaining said extension bar in a predetermined length; and
said distal anchor assembly cuff restraint includes a ratcheting fastener with multiple teeth for adjusting a distal anchor assembly cuff restraint opening size and retaining said cuff on the wrist.

11. A method of stabilizing a patient's arm and restricting elbow and wrist movement, which method comprises the steps of:
providing a proximal anchor assembly including a proximal anchor strap configured for encircling the patient's upper arm and a bicep pad connected to said proximal anchor strap;
securing said proximal anchor assembly to the patient's upper arm;
providing a distal anchor assembly including a cuff configured for encircling the patient's wrist and restraining the wrist against movement;
securing said distal anchor assembly to the patient's wrist;
providing an extension bar with a proximal end pivotally connected to said bicep pad and a distal end pivotally connected to said distal anchor assembly; and
maintaining the patient's arm in a predetermined position defined by said proximal anchor assembly, said distal anchor assembly and said extension bar.
